(19) United States
(12) Patent Application Publication
(10) Pub. No.: US 2019/0117637 A1
(43) Pub. Date: Apr. 25, 2019

(54) PHARMACEUTICAL COMPOSITIONS AND METHODS FOR TREATMENT OF PAIN

(71) Applicant: Board of Regents of the University of Texas System, Austin, TX (US)

(72) Inventors: Alan FRAZER, San Antonio, TX (US); Daniel J. LODGE, San Antonio, TX (US)

(73) Assignee: Board of Regents of the University of Texas System, Austin, TX (US)

(21) Appl. No.: 16/309,733

(22) PCT Filed: Jun. 13, 2017
(86) PCT No.: PCT/US17/37229

§ 371 Date: Dec. 13, 2018

Related U.S. Application Data
(60) Provisional application No. 62/349,531, filed on Jun. 13, 2016.

(51) Int. Cl. A61K 31/4468; A61K 31/554; A61K 31/485; A61K 31/519; A61K 31/167; A61P 25/04

(52) U.S. Cl. CPC A61K 31/4468; A61K 31/554; A61P 25/04; A61K 31/519; A61K 31/167; A61K 31/485

(57) ABSTRACT

Compositions and methods for treatment of acute and chronic pain are disclosed herein. These compositions include fixed dose oral formulations containing an atypical antipsychotic drug and an opioid. Also provided here are methods of treatment of pain, particularly chronic pain while attenuating the abuse-liability of prescription pain medications.

### TECHNICAL FIELD

Disclosed herein are compositions and methods for treatment of acute and chronic pain, while attenuating the abuse-liability of prescription pain medications.

### BACKGROUND

Prescription drugs are, after marijuana, the most commonly abused substances in the United States. Even patients who take pain medications for legitimate reasons may become addicted. Indeed, it is estimated that as many as 1 in 4 patients using opioid-based management for cancer pain become addicted to their medications. Moreover, given the costs of prescription drugs, many patients are turning to heroin, with an estimated 1 in 15 people who take prescription pain relievers trying heroin within 10 years. This has resulted in an epidemic of significant public health concern. According to Justice Department numbers, 52,000 people died from drug overdoses in 2015. More than half those deaths involved the use of heroin, the synthetic pain medication fentanyl and other opioid drugs. One of the shortcomings in the field is the lack of compositions that continue managing the pain while reducing the abuse liability of the prescription pain medications, with such abuse sometimes resulting in overdose and death from respiratory depression.

### SUMMARY

Disclosed herein are compounds and methods addressing the shortcomings of the art, and may provide any number of additional or alternative advantages. Described herein are compounds, formulations, and methods for treatment of either acute or chronic pain, while attenuating the abuse-liability of prescription pain medications. Embodiments described herein include a pharmaceutical composition containing a fixed dose oral formulation of an atypical antipsychotic drug and an opioid. Embodiments described here include fixed dose formulations containing an antagonist or partial agonist of D2 dopamine receptors, such as an atypical antipsychotic drug, and a prescription pain medication. Specific embodiments include a pharmaceutical composition containing an atypical antipsychotic drug and an opioid as a single oral formulation. Embodiments also include pharmaceutically acceptable derivatives of the atypical antipsychotic drug and the opioid.

Embodiments of the invention include a therapeutically effective fixed dose pharmaceutical composition containing an atypical antipsychotic drug; and an opioid, or pharmaceutically acceptable derivatives thereof. In certain embodiments, the amount of atypical antipsychotic drug present is in an approximate range of 2.5-30 mg (olanzapine equivalent doses). In certain embodiments, the amount of the opioid present is in an approximate range of 10-200 mg (morphine equivalent doses). In certain embodiments, the opioid is oxycodone and the atypical antipsychotic drug is risperidone. In certain embodiments, the opioid is oxycodone and the atypical antipsychotic drug is ziprasidone. In certain embodiments, the opioid is fentanyl and the atypical antipsychotic drug is quetiapine. The pharmaceutical composition can be a single tablet for oral consumption. In certain embodiments, the pharmaceutical composition is a controlled release formulation. The pharmaceutical composition can include one or more inert pharmaceutically acceptable excipients. In certain embodiments, the pharmaceutical composition also includes a non-opioid analgesic drug. The non-opioid analgesic drug can be acetaminophen. In certain embodiments, the atypical antipsychotic drug is selected from the group consisting of aripiprazole, olanzapine, quetiapine, risperidone, and ziprasidone. In certain embodiments, the opioid is selected from the group consisting of hydrocodone, oxycodone, and fentanyl.

Also disclosed herein are methods of treating or reducing pain in a subject and reducing the abuse liability of the pain medication using the pharmaceutical compositions disclosed herein. In certain embodiments, the method includes administering to the subject a therapeutically effective fixed dose pharmaceutical composition containing an atypical antipsychotic drug and an opioid, or pharmaceutically acceptable derivatives thereof. In certain embodiments, the pharmaceutical composition contains from about 2.5 mg to 30 mg (olanzapine equivalent doses) of the atypical antipsychotic drug. In certain embodiments, the pharmaceutical composition contains from about 10 mg to 200 mg (morphine equivalent doses) of the opioid. In certain embodiments, the atypical antipsychotic drug is selected from the group consisting of aripiprazole, olanzapine, quetiapine, risperidone, and ziprasidone. In certain embodiments, the opioid is selected from the group consisting of hydrocodone, oxycodone, and fentanyl. In certain embodiments, the opioid is oxycodone and the atypical antipsychotic drug is risperidone. In certain embodiments, the opioid is oxycodone and the atypical antipsychotic drug is ziprasidone. In certain embodiments, the opioid is fentanyl and the atypical antipsychotic drug is quetiapine.

Numerous other aspects, features and benefits of the present disclosure may be made apparent from the following detailed description taken together with the figures. The pharmaceutical compositions can include compounds described herein, other components, or ingredients depending on desired prevention and treatment goals. It should be further understood that both the foregoing general description and the following detailed description are exemplary and explanatory, and not meant to be limiting, and are intended to provide further explanation of the inventions as claimed.

### BRIEF DESCRIPTION OF THE DRAWINGS

The present disclosure can be better understood by referring to the following figures.

FIG. 1 is graphical representation of the analgesic response to fentanyl (100 μg/kg i.p.) and the response following the co-administration of quetiapine (10 mg/kg i.p.).

FIG. 2 is a graphical representation of the decreased reinforcing effect of heroin (32 μg/kg/infusion) following the administration of quetiapine (10 mg/kg i.p.).

FIG. 3 is a graphical representation of the respiratory depression in response to fentanyl (20 μg/kg i.p.) and the response following the co-administration of quetiapine (10 mg/kg i.p.).

FIG. 4 is a graphical representation of the analgesic response to oxycodone alone (2 mg/kg i.p.), and the response following the co-administration of risperidone (0.3 mg/kg i.p.) and ziprasidone (3.0 mg/kg i.p.).

### DETAILED DESCRIPTION

Reference will now be made to the exemplary embodiments illustrated in the figures, and specific language will be used here to describe the same. It will nevertheless be understood that no limitation of the scope of the disclosure is thereby intended. Alterations and further modifications of the inventive features illustrated here, and additional applications of the principles of the disclosures as illustrated here, which would occur to one skilled in the relevant art and having possession of this disclosure, are to be considered within the scope of the disclosure.

Current research implicates mesolimbic dopamine transmission in the reinforcing effects of most drugs of abuse, with such effects leading to their abuse liability. A wide variety of pharmacologically distinct abused drugs have been demonstrated to increase dopamine in the nucleus accumbens. And, this is true for CNS stimulants (such as amphetamine and cocaine), depressants (such as alcohol and benzodiazepines), and hallucinogens (such as LSD and PCP) as well as for opioids. The mechanisms by which opioids increase dopamine release in terminal regions has been examined previously and demonstrated to be attributable to disinhibition of the dopamine neurons located in the ventral tegmental area (VTA). Opioid-induced activation of μ-opioid receptors decreases GABA transmission via inhibitory G-protein signaling. Given that dopamine neurons in the VTA receive a tonic inhibitory input from both local and projection GABAergic neurons, an opioid-induced decrease in GABA signaling would augment dopamine neuron activity. This has been confirmed using electrophysiology and microdialysis in rodents. Given this role for dopamine in drug abuse, blockade of dopamine signaling should decrease the reinforcing effects of abused drugs, including opioids. There are numerous studies demonstrating that blocking dopamine receptors (specifically the D2 and D2-like subtypes) is sufficient to decrease the reinforcing effects of most abused drugs. Unfortunately, these preclinical results have failed to be translated to the clinical setting as antipsychotics are ineffective in the treatment psychostimulant dependence. This is likely due to several factors. Even though the euphoric effects of abused drugs, which contributes initially to their abuse potential, are dopamine dependent, effects such as addiction, craving and impulsivity are more complex, involving regions outside of the traditional mesolimbic dopamine system (such as prefrontal and orbitofrontal cortex, hippocampus and amygdala). Perhaps even more importantly, a method to decrease the euphoric effects of abused substances is unlikely to be used by those who choose to abuse such substances. That is one reason why an opioid antagonist such as naltrexone has limited utility in the treatment of those who have elected to take opioids. Embodiments disclosed herein are directed to patients needing pain relief such as that afforded by an opiate but do not want to abuse the opioids or become addicted to them. These compositions also serve patient populations at risk for addiction based on history.

Fixed dose formulations containing an atypical antipsychotic drug (such as D2 receptor antagonists or partial agonists) and a prescription pain medication in the same tablet or capsule are disclosed herein. These formulations are directed to methods of treatment of either acute or chronic pain while attenuating the abuse-liability of prescription pain medications. More particularly, these formulations contain fixed dose oral formulations of an atypical antipsychotic drug and an opioid. Specifically, this fixed dose combination significantly reduces the abuse potential of these commonly prescribed pain medications, by decreasing their reinforcing effects, without decreasing their analgesic properties or augmenting their side effects. This approach is truly transformative and revolutionizes the treatment of pain, particularly chronic pain. By combining opioid-based analgesics with an antipsychotic drug in a fixed dose combination, the euphoric effects of the opioid are blunted. An embodiment of the invention includes a widely prescribed (and abused) opioid, such as oxycodone, hydrocodone, or fentanyl in combination with an antipsychotic. An embodiment of the invention includes an atypical antipsychotic, such as aripiprazole, quetiapine, olanzapine, risperidone, or ziprasidone, as it produces less extrapyramidal movement symptoms than the original antipsychotic drugs. Embodiments disclosed herein also other modulators of dopamine D2 and D2-like receptors. Embodiments disclosed herein also include dopamine receptor antagonists, partial agonists, inverse agonists, and allosteric modulators. As with the majority of abused substances, the rewarding properties of opioids are thought to reside in their ability to increase dopamine release in areas of the brain such as the nucleus accumbens. By blocking dopamine D2 receptors, antipsychotic agents reduce the reinforcing and rewarding properties of many abused drugs, and thus, dramatically reduce the abuse liability of pain medications.

Embodiments of the invention include fixed dose formulations containing an atypical antipsychotic drug and a prescription pain medication. A specific embodiment includes a therapeutically effective fixed dose pharmaceutical composition containing an atypical antipsychotic drug and an opioid, or pharmaceutically acceptable salts thereof. The atypical antipsychotic drug can be present in an approximate range of 2.5-30 mg (olanzapine equivalent doses). The amount of the opioid can be present in an approximate range of 10-200 mg (morphine equivalent doses). The pharmaceutical composition can be a single tablet or capsule for oral consumption. The pharmaceutical composition can be a controlled release formulation. The pharmaceutical composition can further contain one or more inert pharmaceutically acceptable excipients. Embodiments also include an antagonist or partial agonist of dopamine D2 and D2-like receptors, in combination with an opioid and an additional analgesic drug (such as acetaminophen). An embodiment of the invention includes a method for treating or reducing pain in a subject while substantially reducing the abuse liability of the pain-relieving medication. The method includes administering to the subject a therapeutically effective fixed dose pharmaceutical composition containing an atypical antipsychotic drug and a prescription pain medication. An embodiment of the invention includes administering to the subject a therapeutically effective fixed dose pharmaceutical composition containing an atypical antipsychotic drug and an opioid. Examples of atypical antipsychotic drugs are aripiprazole, olanzapine, quetiapine, risperidone, ziprasidone, or a combination thereof. Examples of the opioid can be hydrocodone, oxycodone, fentanyl, or a combination thereof.

As used here, the following terms may have the following definitions:

A “pharmaceutical composition” refers to a mixture of two or more of the compounds described herein, or a pharmaceutically acceptable salt, solvate, hydrate or prodrug thereof as an active ingredient. The pharmaceutical composition can also include at least one pharmaceutically acceptable carrier or excipient. The purpose of the pharmaceutical composition is to facilitate administration of the fixed dose combinations to a subject. In some embodiments, the pharmaceutical composition includes two or more pharmaceutically acceptable carriers and/or excipients.

The term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the active ingredient. For example, a pharmaceutically acceptable derivative thereof of an atypical antipsychotic drug includes all derivatives of the atypical antipsychotic drug (such as salts, pro-drugs, metabolites, esters, ethers, hydrates, polymorphs, solvates, complexes, and adducts) which, upon administration to a subject, are capable of providing (directly or indirectly) the atypical antipsychotic drug. For example, a pharmaceutically acceptable derivative thereof of an prescription pain medication includes all derivatives of the prescription pain medication (such as salts, pro-drugs, metabolites, esters, ethers, hydrates, polymorphs, solvates, complexes, and adducts) which, upon administration to a subject, are capable of providing (directly or indirectly) the prescription pain medication to a subject.

As used herein, the term “pharmaceutically acceptable salt” refers to those salts of a compound, which retain the biological effectiveness and properties of the parent compound. And unless otherwise indicated, a pharmaceutically acceptable salt includes salts of acidic or basic groups, which may be present in the compounds disclosed herein. The present disclosure also relates to a process for the preparation of the above pharmaceutically acceptable salts, their derivatives, their analogs, their tautomeric forms, their stereoisomers, their polymorphs, and pharmaceutical compositions containing them.

Certain embodiments relate to pharmaceutically acceptable salts formed by the compounds described herein, their derivatives, their analogs, their tautomeric forms, their stereoisomers, their polymorphs and pharmaceutically acceptable compositions containing them. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methyl benzoate, o-acetoxybenzoate, naphthalene-2-benzoate, bromide, isobutyrate, phenyl butyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

Embodiments of the invention include pharmaceutical compositions containing an atypical antipsychotic drug and a prescription pain medication, or a pharmaceutically acceptable salt thereof, and a pharmaceutically acceptable carrier or excipient. Pharmaceutically acceptable ingredients, such as excipients, diluents, fillers, binders, and carriers can be inert or actively contribute to the delivery and distribution of the compounds described herein. The formulations used in embodiments herein include excipients, such as microcrystalline cellulose, lactose monohydrate, hydroxypropyl cellulose, croscarmellose sodium and magnesium stearate, preferably at least about 50 wt %, such as in the range from about 50% to about 95 wt %, including the range from about 50-90 wt %, and more preferably in the range from about 55-85 wt %, such as in the range from about 60% to about 85 wt %, or in the range from about 65 wt % to about 80 wt %, including about 60 wt %, about 65 wt %, about 70 wt %, about 75 wt %, or about 80 wt %.

As used herein, the term “prescription pain medication” refers to opioid compounds that are administered to an animal to mitigate pain associated with disease or injury or medical procedures. The amount of the opioid can be present in an approximate range of 10-200 mg (morphine equivalent doses). In certain embodiments, the amount of the opioid can be present in an approximate range of 20-200 mg (morphine equivalent doses), or about 50-150 mg (morphine equivalent doses), or about 20-1500 mg (morphine equivalent doses), or about 20-100 mg (morphine equivalent doses). This includes: (2-Phenylethyl)-4-phenyl-4-acetoxypiperidine, [1-(2-Hydroxy-2-phenylethyl)-3-methyl-4-piperidyl]-Nphenylpropanamide, [3-Methyl-1-[2-(2-thienyl)ethyl)-4-piperidyl]-N-phenyl prop, 1-Benzyl-4-phenyl-4-propionoxypiperidine, 1-Methyl-4-phenyl-4-propionoxypiperidine, 2-Ethyl-5-methyl-3,3-diphenyl-1-pyrroline, Acetylcodeine, Acetylmethadol, Acetylmorphine, Acetyl-N,N-dinormethadol, Acetylnormethadol, Alfentanil, Allylnormetazocine, Benzylidine-7-dehydronaltrexone, Bremazocine, Buprenorphine, Buprenorphine-3-P-D-glucuronide, Butorphanol, Carfentanil, Clocinnamox, Codeine, Codeine-6-P-D-glucuronide, Cyclazocine, DAMGO, Deltorphin, Demerol, Dextromoramide, Diacetylmorphine, Dihydrohydroxycodeinone, Dimethyl-3,3-d iphenyl-2-ethylpyrrolinium, Dimethyl-3,3-diphenyl-2-pyrrolidone, Dimethylamino-2,2-diphenylvaleric acid, Dimethylamino-2,2-diphenylvaleronitrile, Dinormethadol, Diphenoxylate, Diphenyl-2-ethyl-5-methyl-1-pyrroline, Diprenorphine, DPDPE, DSLET, Ethyl-1,5-dimethyl-3,3-diphenylpyrrolinium perchlorate, Ethyl-5-methyl-3,3-diphenyl-1-pyrroline hydrochloride, Ethylketazocine, Etonitazene, Etorphine, Fentanyl, Funaltrexamine, GNTI, hydrocodone, Hydromorphone, Hydroxymethadone, IBNtxA, Isocodeine, Isomorphine, J-1 13397, JDTic, Levorphanol, LY-2456302, Meperidine, Metazocine, Methadol, Methadone, Methoxymorphinan, Methylfentanyl, Morphine, Morphine-6-glucuronide, Nalbuphine, Nalfurafine, Nalorphine, Naloxol, Naloxone, Naltrexamide, Naltrexamine, Naltrexol, Naltrexone, Naltriben, Naltrindole, Norbinaltorphimine, Norbuprenorphine, Norcodeine, Normeperidine, Normetazocine, Normethadol, Normorphine, Noroxymorphone, Norpropoxyphene, N-Phenyl-N-[1-(2-thienyl)methyl-4-piperidyl] propanamide, Oxycodone, Oxymorphone, Pentazocine, Phenazocine, Piperidyl-N-phenylpropanamide, Propionylnorpropoxyphene, Propoxyphene, Ro 64-6198, Salvinorin A, SB-612111, SNC 80, Spiradoline, Sufentani 1, Thebaine, U50,488, U69,593.

As used herein, the term “antipsychotic drug” refers to any compound(s) that is administered to an animal to decrease dopamine D2 and D2-like receptor signaling. The atypical antipsychotic drug can be present in an approximate range of 2.5-30 mg (olanzapine equivalent doses). In certain embodiments, the atypical antipsychotic drug can be present in an approximate range of 5-30 mg (olanzapine equivalent doses) or about 5-25 mg (olanzapine equivalent doses) or about 10-30 mg (olanzapine equivalent doses). This includes: A-381393, AJ76, Amisulpride, Apomorphine, Aripiprazole, Asenapine, Blonanserin, BP 897, Brexpiprazole, Bromocriptine, Butaclamol, Cabergoline, Cariprazine, Carpipramine, Chlorpromazine, Chlorprothixene, Clocapramine, Clozapine, CP-226269, CP-293019, Oomperidone, Oroperidol, Eticlopride, FAUC213, Flupentixol, Fluphenazine, Haloperidol, Iloperidone, L-741,626, L741742, L745870, L-750,667, Levomepromazine, Lisuride, Loxapine, Lurasidone, Melperone, Mesoridazine, Metoclopramide, ML321, ML398, Molindone, Mosapramine, Nafadotride, Nemonapride, NGB 2904, NGD941, N-Methylspiperone, Olanzapine, Paliperidone, PD 1 68,077, Pergolide, Periciazine, Perospirone, Perphenazine, PGO1037, Pimozide, Pipotiazine, Piribedil, Prochlorperazine, Promazine, Quetiapine, Raclopride, RBI257, Remoxipride, Risperidone, Ro 10-4548, Roxindole, 5-14297, 533084, SB 277011-A, SCH-23390, Sertindole, Sonepiprazole, Spiperone, Stepholidine, Sulpiride, Terguride, Thioproperazine, Thioridazine, Thiothixene, Trans-Flupenthixol, Trifluoperazine, U 101958, UH232, Ziprasidone, Zotepine, Zuclopenthixol.

As used herein, unless otherwise noted, the terms “treating”, “treatment” and the like, shall include the management and care of a subject or patient (preferably mammal, more preferably human) for the purpose of combating a disease, condition, or disorder and includes the administration of the compounds of the present disclosure to prevent the onset of the symptoms or complications or alleviate the symptoms or complications.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes a reduction in symptoms and attenuation of abuse. Compositions for administration herein may form solutions, suspensions, tablets, pills, capsules, sustained release formulations or powders. Illustratively, an effective amount of the compositions described here can range from nanogram/kg to microgram/kg to milligram/kg amounts for young children and adults. Equivalent dosages for lighter or heavier body weights can readily be determined. The dose should be adjusted to suit the individual to whom the composition is administered and will vary with age, weight and metabolism of the individual. The exact amount of the composition required will vary from subject to subject, depending on the species, age, weight and general condition of the subject, the particular atypical antipsychotic drug and a prescription pain medication used, its mode of administration and the like. An appropriate amount can be determined by one of ordinary skill in the art using only routine experimentation given the teachings herein. One skilled in the art will realize that dosages are best optimized by the practicing physician or veterinarian and methods for determining dose amounts and regimens and preparing dosage forms are described, for example, in Remington: The Science and Practice of Pharmacy, 22nd edition or Goodman & Gilman's The Pharmacological Basic of Therapeutics, 12^{th} edition; the Merck Manual, Professional Version.

The term “combined use” or “combination” means that the individual components can be administered simultaneously, such as in the form of a single formulation of fixed dose. The combination of an atypical antipsychotic drug and a prescription pain medication can be employed in admixtures with conventional excipients, i.e., pharmaceutically acceptable organic or inorganic carrier substances suitable for oral administration, known to the art. Suitable pharmaceutically acceptable carriers include but are not limited to alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, and polyvinylpyrrolidone. The pharmaceutical preparations can be sterilized and, if desired, mixed with auxiliary agents, e.g., lubricants, preservatives, stabilizers, wetting agents, emulsifiers, salts for influencing osmotic pressure buffers, coloring, flavoring and/or aromatic substances and the like. For oral administration, particularly suitable are tablets, capsules, caplets and gelcaps. The compositions intended for oral use may be prepared according to any method known in the art and such compositions may contain one or more agents selected from the group consisting of inert, non-toxic pharmaceutically excipients, which are suitable for the manufacture. These compositions may be uncoated or coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release the atypical antipsychotic drug and the prescription pain medication in desired areas of the gastro-intestinal (GI) tract, e.g., the stomach or small intestine, such that an absorption profile is provided, which is capable of providing at least about 2 hours and preferably about 4 to up to about twenty-four hours of analgesia to a patient. It is also possible to formulate compositions which release a portion of the dose in one desired area of the GI tract, e.g., the stomach, and release the remainder of the dose in another area of the GI tract, e.g., the small intestine.

The term “fixed dose” means two or more active pharmaceutical ingredients are provided as a predetermined combination of predetermined doses of the two or more active pharmaceutical ingredients. Embodiments of the invention include composition and methods of administering fixed dose combinations of antipsychotic drug with an opioid receptor agonist. Such a combination does not alter the beneficial effects of the opioid medication. The analgesic properties of opioid medications with and without a fixed dose combination of an antipsychotic agent were examined. For example, mu-opioid receptors are present at the terminals of primary afferent C-fibers where they act to decrease neurotransmitter release via Gᵢ-mediated inhibition of Ca²⁺ channels. These mu-opioid receptors are also present on second order neurons within the spinal cord where stimulation leads to hyperpolarization due to activation of GIRK channels. Thus, opioids decrease the transmission of pain signals from the periphery to the CNS. In addition, mu-opioid receptors in the periaqueductal grey have been implicated in the analgesic properties of opioids. These regions are distinct from those implicated in the mediation of the reinforcing effects of these drugs. Thus, it is unlikely that dopamine receptor blockade, by antipsychotic co-administration, will interfere with the analgesic properties of opioids, and the data provided herein aligns with this hypothesis. The atypical antipsychotic drug can be present in an approximate range of 2.5-30 mg (olanzapine equivalent doses). The amount of the opioid can be present in an approximate range 10-200 mg (morphine equivalent doses).

Embodiments of the invention include composition and methods of administering fixed dose combinations of oxycodone and an atypical antipsychotic drug. Embodiments of the invention include composition and methods of administering fixed dose combinations of oxycodone and olanzapine. Embodiments of the invention include composition and methods of administering fixed dose combinations of oxycodone and aripiprazole. Embodiments of the invention include composition and methods of administering fixed dose combinations of oxycodone and quetiapine. Embodiments of the invention include composition and methods of administering fixed dose combinations of oxycodone and risperidone. Embodiments of the invention include composition and methods of administering fixed dose combinations of oxycodone and ziprasidone.

Embodiments of the invention include composition and methods of administering fixed dose combinations of hydrocodone and an atypical antipsychotic drug. Embodiments of the invention include composition and methods of administering fixed dose combinations of hydrocodone and olanzapine. Embodiments of the invention include composition and methods of administering fixed dose combinations of hydrocodone and aripiprazole. Embodiments of the invention include composition and methods of administering fixed dose combinations of hydrocodone and quetiapine. Embodiments of the invention include composition and methods of administering fixed dose combinations of hydrocodone and risperidone. Embodiments of the invention include composition and methods of administering fixed dose combinations of hydrocodone and ziprasidone.

Embodiments of the invention include composition and methods of administering fixed dose combinations of fentanyl and an atypical antipsychotic drug. Embodiments of the invention include composition and methods of administering fixed dose combinations of fentanyl and olanzapine. Embodiments of the invention include composition and methods of administering fixed dose combinations of fentanyl and aripiprazole. Embodiments of the invention include composition and methods of administering fixed dose combinations of fentanyl and quetiapine. Embodiments of the invention include composition and methods of administering fixed dose combinations of fentanyl and risperidone. Embodiments of the invention include composition and methods of administering fixed dose combinations of fentanyl and ziprasidone.

Embodiments of the invention include composition and methods of administering fixed dose combinations of an opioid pain medication and an atypical antipsychotic drug. Embodiments of the invention include composition and methods of administering fixed dose combinations of an opioid pain medication and aripiprazole. Embodiments of the invention include composition and methods of administering fixed dose combinations of an opioid pain medication and olanzapine. Embodiments of the invention include composition and methods of administering fixed dose combinations of an opioid pain medication and quetiapine. Embodiments of the invention include composition and methods of administering fixed dose combinations of an opioid pain medication and risperidone. Embodiments of the invention include composition and methods of administering fixed dose combinations of an opioid pain medication and ziprasidone.

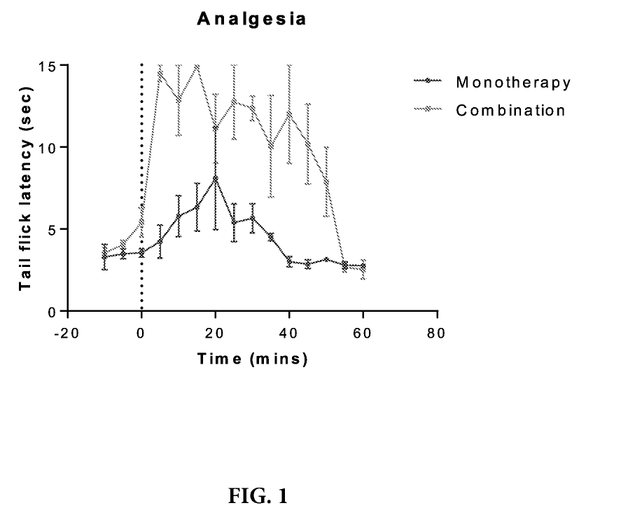

While the ability to reduce the abuse potential of opioid prescription pain medications is paramount, it is equally important that the analgesic properties of the drug remain. Given that the analgesic properties of opioids occur at regions outside of the mesolimbic dopamine system (i.e. periaqueductal grey, medulla, and peripheral neurons), the addition of an antipsychotic is unlikely to interfere with the analgesic properties of the opioid. Dopamine receptor blockade, by antipsychotic co-administration, will most likely not interfere with the analgesic properties of opioids, as indicated at least by the data provided in FIG. 1. Interestingly, these data indicate the opposite effect, i.e. the co-administration of an atypical antipsychotic drug may actually enhance the analgesic effects of a prescription pain medication, such as fentanyl. Thus, a fixed dose combination with an antipsychotic will potentially reduce the abuse liability of the prescription pain medication, such as an opioid, but it may also enhance their analgesic effects, resulting in lower effective doses of the opioid.

Another concern of combination formulations is whether the addition of an atypical antipsychotic drug will increase the severity of opioid-induced side effects. Atypical antipsychotic drugs do produce side effects. In contrast to older agents such as chlorpromazine or haloperidol, they are less likely to produced extrapyramidal motoric effects such as Parkinsonian symptoms or tardive dyskinesias. But, their chronic use is associated with weight gain and in some patients the development of type II diabetes. In spite of such side effects, they are used widely to control psychotic symptoms such as those in schizophrenia or bipolar disorder. A major side effect of opioid-based analgesics is respiratory depression and is the major cause of death for opioid overdose. Indeed, deaths associated with prescription opioid use have skyrocketed and at least half of all opioid overdose deaths involve a prescription opioid. Given that antipsychotics are central nervous system depressants; it may appear that a combination with an opioid may be contraindicated. However, it should be noted that central nervous system depression is not equivalent to respiratory depression and none of the first or second generation antipsychotic drugs cause respiratory depression. Thus, a fixed dose combination is not anticipated to increase the severity of opioid-induced respiratory depression and provided here are the confirmatory data.

Other side effects of opioid use include constipation, nausea/vomiting, extrapyramidal symptoms, sedation or other sleep disturbance disorders, and cardiovascular complications.

While not life threatening, opioid-induced constipation is a significant side effect associated with opioid receptor expression in the enteric nervous system, which is comprised of the myenteric and submucosal plexus. By binding to the opioid receptors in the enteric nervous system, endogenous and exogenous opioids are able to modulate gastrointestinal motility and secretion. Opioid based therapeutics therefore induce constipation via GI-mediated inhibition in the enteric nervous system. Dopamine neurons are present in both plexuses of the bowel with dopamine D2 receptors being localized throughout the gastrointestinal system. The net effect of dopamine on gastrointestinal motility is inhibitory, therefore, D2 antagonists would be expected to augment GI motility and decrease the severity of opioid-induced constipation. Antipsychotic drugs are not simply D2 receptor antagonists and clinical studies have demonstrated an increased risk for constipation with antipsychotics (10% of patients). This is attributable to the anticholinergic effects of certain atypical antipsychotics. In certain embodiments, specifically selected antipsychotic drugs with low incidences of anticholinergic effects are selected to be part of the fixed dose combination. Further studies to compare fecal number, weight and water content following a fixed dose combination with that obtained with opioid monotherapy will help determine optimal combinations and dosage forms.

Nausea has been reported to occur in approximately 25% of patients treated with opioid-based analgesics. This can occur via a number of different pathways including direct stimulation of the chemoreceptor trigger zone, located in the area postrema of the medulla. Similarly, dopamine receptors have been implicated in the control of nausea and vomiting where dopamine D2 receptors predominate. Antipsychotic drugs and related agents (i.e. metoclopramide) are routinely used to combat opioid-induced nausea. Thus, a fixed dose combination of an antipsychotic and opioid-analgesic would likely protect against opioid-induced nausea and vomiting.

All antipsychotic drugs are dopamine D2-like receptor antagonists or partial agonists and produce clinically significant effects by reducing dopaminergic transmission through D2 receptors. Unfortunately, they also cause dopamine receptor blockade in the nigro-striatal dopamine system which is the cause of debilitating extrapyramidal side effects including Parkinson's-like symptoms (tremors, muscle rigidity, tardive dyskinesia). Embodiments of the invention therefore include atypical or 2nd generation antipsychotic drugs, which are known to display reduced extrapyramidal side effects when compared to the first generation drugs.

High doses of antipsychotic drugs have been known to induce sedative effects, whereas low-potency drugs (i.e. chlorpromazine) are more sedating than the high-potency antipsychotics, such as haloperidol. These drugs also have the potential to disturb sleep/wake cycles. Embodiments of the invention therefore include atypical antipsychotic drugs at specifically selected doses that do not appear to lead to marked sedation. Nonetheless, given that opioids also display sedative properties at high doses, it is important to examine whether a fixed dose combination will potentiate these effects. Further studies can be conducted to examine cortical EEG as an index of sedation, by examining slow-wave activity, and thus determining the optimal combinations and dosage forms.

A majority of antipsychotic drugs produce quantitative changes in the electrocardiogram known as QTc prolongation. This is reflective of an increased time to repolarize the ventricles and increases the likelihood of developing ventricular arrhythmias known as torsades de pointes, TdP. The mechanism by which this occurs is through blockade of human ether-a-go-go-related gene (hERG) K+ channels on the cardiac myocytes. This is a potential serious side effect; however, the majority of FDA approved antipsychotics display only minor QTc prolongation and the risks of TdP are diminished with low doses of second generation antipsychotics. Thus, it is unlikely that a fixed-dose combination of an opioid and an antipsychotic will appreciably effect QTc. Further studies can be conducted to examine potential cardiovascular alterations for example using implantable telemetry.

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent.

### Example 1

To examine the analgesic properties of the fixed dose combination of an opioid with an antipsychotic drug, the standard tail-flick latency task was used, in which a rat's tail is placed in a hot (about 52.5° C.) water bath and the latency in removing the tail is measured. A robust analgesic effect of fentanyl (100 μg/kg i.p.) was measured, as determined by an increased latency for the tail flick. Interestingly, the analgesic response was actually enhanced by a fixed dose combination of fentanyl (100 μg/kg i.p.) and quetiapine (10 mg/kg i.p.) as shown in FIG. 1.

### Example 2

Embodiments of the invention include an analgesically effective fixed dose pharmaceutical composition that attenuates the abuse potential of the analgesic component. Although a variety of procedures can be used to assess a drug's abuse liability, intravenous (IV) self-administration procedures, in which drug infusions are delivered contingent upon an animal making a response (e.g., pressing a lever), are the gold-standard in the field as they have high levels of both face and predictive validity. Using IV self-administration procedures, it has been consistently demonstrated that opioid-based drugs are highly effective reinforcers in rats, i.e., they are self-administered, consistent with their misuse and abuse by humans.

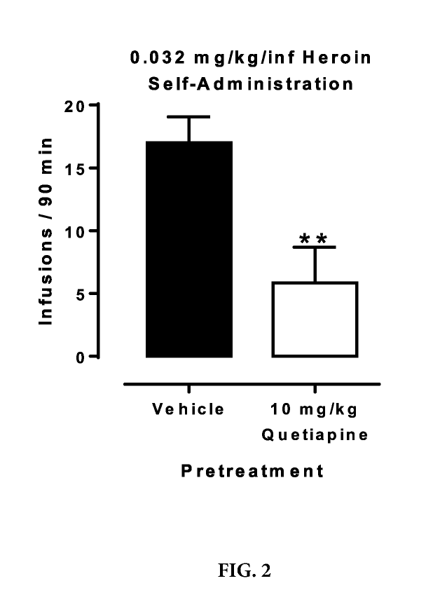

In this experiment, prior to initiating self-administration studies, rats were surgically prepared with an indwelling catheter in the left femoral vein under isoflorane anesthesia. Catheters were flushed daily with 0.5 ml of heparinized saline (100 U/ml) to promote patency, and with methohexital (3.2 mg/kg; IV) once weekly (and as needed) to test catheter patency. All self-administration studies were conducted in standard operant chambers equipped with two response levers, stimulus lights, and an infusion pump, as previously described. Rats were trained to lever press for i.v. heroin infusions (0.032 mg/kg/infusion) under a fixed dose schedule wherein five active lever presses resulted in heroin delivery. Rats then were tested for heroin self-administration after pretreatment with 10 mg/kg quetiapine prior to testing sessions. Data in FIG. 2 demonstrates that pretreatment with the atypical antipsychotic quetiapine (10 mg/kg i.p.) decreased the self-administration of heroin, an opioid agonist. This data demonstrates that the magnitude of self-administration of an opioid alone will be significantly attenuated when given in a fixed dose combination with an atypical antipsychotic drug.

### Example 3

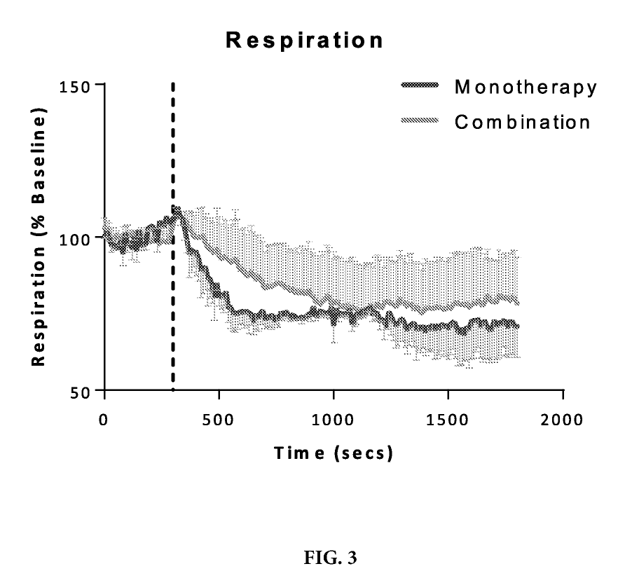

Respiratory rate was recorded in chloral hydrate sedated rats via a pressure transducer placed on the sternum. As shown in FIG. 3, the respiratory depression observed with fentanyl (20 μg/kg i.p.) was not increased by its co-administration with quetiapine (10 mg/kg i.p.). While fentanyl (20 μg/kg i.p.) produced the anticipated decrease in respiratory rate, the fixed dose combination of fentanyl (20 μg/kg i.p.) and quetiapine (10 mg/kg i.p.) produced a similar decrease in respiration as that seen with fentanyl alone suggesting that this combination does not potentiate this side effect of opioid medications. The D2 receptor antagonists may actually be beneficial in cases of hypoxia and hypercapnia (i.e. following opioid induced respiratory depression) where augmentation of carotid chemoreceptor and ventilator response would be valuable. Further studies can be carried out to measure respiratory function using implantable telemetry which will also provide information regarding activity, body temperature and central nervous system depression (via cortical EEG).

### Example 4

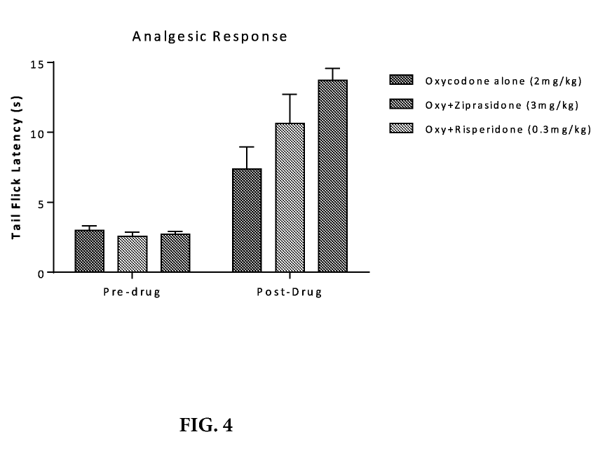

Analgesia was determined by the tail-flick latency task as described in Example 1. Rats were lightly held and their tail immersed ˜2 cm into a water bath heated to 52.5° C. The latency to remove the tail from the water was recorded before (pre-drug) and 30 mins following (post-drug) drug administration. If the rat did not remove its tail within 15 seconds, the trial was stopped to prevent tissue damage. The drugs examined were oxycodone alone (2 mg/kg i.p.), oxycodone plus risperidone (0.3 mg/kg i.p.) and oxycodone plus ziprasidone (3.0 mg/kg i.p.). The average and median tail flick latency pre-drug treatment was about 2.5-2.9 seconds and 2.7-3 seconds. Following treatment with oxycodone alone, the average and median tail flick latency pre-drug treatment was about 7.3 seconds and 5.8 seconds respectively. Following treatment with oxycodone and risperidone, the average and median tail flick latency pre-drug treatment was about 10.6 seconds and 15 seconds respectively. Following treatment with oxycodone and ziprasidone, the average and median tail flick latency pre-drug treatment was about 13.7 seconds and 15 seconds respectively. Results from this experiment, as shown in FIG. 4 demonstrated that the addition of an atypical antipsychotic increased the analgesic effect of oxycodone as determined by an increase in tail-flick latency in rats.

Whenever a numerical range is indicated herein, it is meant to include any cited numeral (fractional or integral) within the indicated range. It is understood that the examples and embodiments described herein are for illustrative purposes only and that various equivalents, modifications or changes in light thereof will be suggested to persons skilled in the art and are to be included within the spirit and purview of this application and scope of the appended claims.

1. A therapeutically effective fixed dose pharmaceutical composition comprising: an atypical antipsychotic drug; and an opioid, or pharmaceutically acceptable derivatives thereof.

2. The pharmaceutical composition of claim 1, wherein the composition contains from about 2.5 mg to 30 mg (olanzapine equivalent doses) of the atypical antipsychotic drug.

3. The pharmaceutical composition of claim 1, wherein the composition contains from about 10 mg to 200 mg (morphine equivalent doses) of the opioid.

4. The pharmaceutical composition of claim 1, wherein pharmaceutical composition is a single tablet for oral consumption.

5. The pharmaceutical composition of claim 4, wherein pharmaceutical composition is a controlled release formulation.

6. The pharmaceutical composition of claim 1, further comprising one or more inert pharmaceutically acceptable excipients.

7. The pharmaceutical composition of claim 1, further comprising a non-opioid analgesic drug.

8. The pharmaceutical composition of claim 7, wherein the non-opioid analgesic drug is acetaminophen.

9. The pharmaceutical composition of claim 1, wherein the atypical antipsychotic drug is selected from the group consisting of aripiprazole, olanzapine, quetiapine, risperidone, and ziprasidone.

10. The pharmaceutical composition of claim 1, wherein the opioid is selected from the group consisting of hydrocodone, oxycodone, and fentanyl.

11. A method for reducing pain in a subject and reducing the abuse liability of an opioid, the method comprising
administering to the subject a therapeutically effective fixed dose pharmaceutical composition containing an atypical antipsychotic drug and an opioid, or pharmaceutically acceptable derivatives thereof.

12. The method of claim 11, wherein the pharmaceutical composition contains from about 2.5 mg to 30 mg (olanzapine equivalent doses) of the atypical antipsychotic drug.

13. The method of claim 11, wherein the pharmaceutical composition contains from about 10 mg to 200 mg (morphine equivalent doses) of the opioid.

14. The method of claim 11, wherein the atypical antipsychotic drug is selected from the group consisting of aripiprazole, olanzapine, quetiapine, risperidone, and ziprasidone.

15. The method of claim 11, wherein the opioid is selected from the group consisting of hydrocodone, oxycodone, and fentanyl.

16. The method of claim 11, wherein the opioid is oxycodone and the atypical antipsychotic drug is risperidone.

17. The method of claim 11, wherein the opioid is oxycodone and the atypical antipsychotic drug is ziprasidone.

18. The method of claim 11, wherein the opioid is fentanyl and the atypical antipsychotic drug is quetiapine.

19. The method of claim 11, wherein the pharmaceutical composition further contains a non-opioid analgesic drug.

20. The method of claim 19, wherein the non-opioid analgesic drug is acetaminophen.
